(19) United States
(12) Patent Application Publication
(10) Pub. No.: US 2020/0383903 A1
(43) Pub. Date: Dec. 10, 2020

(54) SUPPLEMENTS COMPOSITION WITH EFFERVESCENT AGENT

(71) Applicant: Life Boost Inc., Plymouth, MI (US)

(72) Inventors: Ted Matthew MILLS, Novi, MI (US); Jeffrey Thomas LINTON, Ann Arbor, MI (US); Chase Ryan LINTON, Ann Arbor, MI (US); Justin Michael GAUVIN, Waltham, MA (US); Melissa Nicole DOBIES, Wixom, MI (US)

(21) Appl. No.: 16/893,768

(22) Filed: Jun. 5, 2020

Related U.S. Application Data
(60) Provisional application No. 62/858,124, filed on Jun. 6, 2019.

(51) Int. Cl. A61K 9/46; A23L 33/17; A23L 33/16; A23L 33/15; A23L 33/12; A23L 33/21; A23P 10/30; A61K 9/48

(52) U.S. Cl. CPC A61K 9/0007; A23L 33/17; A23L 33/16; A61K 9/4891; A23L 33/12; A23L 33/21; A23P 10/30; A23L 33/15

(57) ABSTRACT

A pod for dispensing a nutritional supplement, and a method if providing a nutritional supplement are provided. The pod has a housing defining a plurality of serving chambers arranged in an annular array and surrounding a drive hub. A nutritional supplement is provided within at least one of the plurality of serving chambers. The nutritional supplement has at least one active ingredient and an effervescent agent to form a gas when the nutritional supplement is dispensed from the pod and dissolved in a liquid. At least one sealing member is connected to the housing and extends over the plurality of serving chambers. The at least one sealing member is removable to open a selected one of the plurality of serving chambers to dispense supplement therein.

### CROSS-REFERENCE TO RELATED APPLICATIONS

This application claims the benefit of U.S. provisional application Ser. No. 62/858,124 filed Jun. 6, 2019, the disclosure of which is hereby incorporated in its entirety by reference herein.

### TECHNICAL FIELD

Various embodiments relate to a composition and a system and a method for the delivery of custom-formulated powder-form and/or granulated and/or oil form and/or beadlet-form and/or liquid dietary formulas and/or pharmaceuticals in measured doses or any other ingestible material that may be fit into the serving chamber.

### BACKGROUND

The traditional market for the manufacture and intake of dietary supplements are most often produced but not limited to a tablet, pill, gummy or capsule form. Pills and capsules are difficult for many people to swallow and/or digest. Manufacturing of such dietary supplements in pill/tablet form requires the use of fillers and/or binding agents in order to produce a tablet that is solid and has an acceptable shelf life. Manufactured tablets or capsules can be often large which tends to limit the amount of active ingredient content per delivery form and results in some efficacious dosages requiring multiple pills or tablets. Many consumers will avoid or are unable to take large pills, which leaves the consumer with few attractive alternatives.

The dietary supplement industry has tried to address this issue by providing rapidly dissolving tablets, gummies and chewable tablets. Dietary supplements in dissolving tablet, gummies or chewable form have many of the same negative attributes of capsules and tablets, such as they typically contain fillers, sugars or binding agents which limit the amount of active ingredient content. The excessive use of fillers and binding agents resists digestion in the human (or animal) body; numerous studies have concluded that pill-form vitamins with even moderate amounts of fillers and/or binding agents can pass through the human digestive system with only a fraction of the active ingredients having been absorbed in the body. Gel-type tablets have been developed to help address the absorption issues, but tend to be even larger and more difficult to swallow especially for those who suffer with esophageal dysphagia. These large form factors can adversely influence the behavior of a consumer resulting in non-adherence issues.

Swallowing large pills, and even small pills for some, are difficult for many people. Those who are elderly, those with throat conditions, and children typically experience the most discomfort ingesting pill/tablet form dietary supplements. And in addition to humans, many conscientious pet owners would like to provide dietary supplements to their dog or cat or horse or other valued animal. Some pets will resist taking a dietary supplement in pill-form, regardless of pill size. And some animals have a more rapid digestive through-put than humans, making pills with substantial amounts of fillers and binding agents even less effective by passing through the animal's body before a sufficient load of the active ingredients having been absorbed.

Another issue with prior art dietary supplements relates to correct dosing. As many dietary supplements are sold “over-the-counter”, many consumers will form a subconscious understanding that the dietary supplements do not need to be taken with the same high level of care as they might otherwise give to prescription medicines. As an effect of this subconscious belief, the average consumer may not be as concerned about missing a daily dose, or perhaps at the other extreme of taking two doses when only one is recommended. For example, a busy or distracted person might not recall if they had taken their vitamin pill that day. This person might think “No big deal, I will take one tomorrow”. Or they might think, “No big deal, I will take another pill just to be safe”. In both cases, the person runs the risk of either over-dosing or under-dosing their intake of the dietary supplement. Of course, pills boxes and the like have been developed to help organize pill consumption for people, but such are normally used for prescription medicines only and require a high degree of discipline to use regularly.

### SUMMARY

Various embodiments of the present disclosure relate to a nutritional supplement composition of including the use of effervescent and/or beadlet technology.

According to an embodiment, a pod for dispensing a nutritional supplement is provided. The pod has a housing defining a plurality of serving chambers arranged in an annular array and surrounding a drive hub. A nutritional supplement is provided within at least one of the plurality of serving chambers. The nutritional supplement has at least one active ingredient and an effervescent agent to form a gas when the nutritional supplement is dispensed from the pod and dissolved in a liquid. At least one sealing member is connected to the housing and extends over the plurality of serving chambers. The at least one sealing member is removable to open a selected one of the plurality of serving chambers to dispense supplement therein.

According to another embodiment, a method of providing a nutritional supplement includes providing a housing defining a plurality of serving chambers arranged in an annular array and surrounding a drive hub. A nutritional supplement is provided into at least one of the plurality of serving chambers of the pod, with the nutritional supplement comprising at least one active ingredient and an effervescent agent. The nutritional supplement is sealed within the at least one of the plurality of serving chambers of the housing by connecting at least one sealing member to the housing to form a pod. The at least one sealing member extends over the plurality of serving chambers and is removable to open a selected one of the plurality of serving chambers to dispense supplement therein.

According to yet another embodiment, a nutritional supplement with effervescent and/or beadlet technology is provided.

According to another embodiment, a supplements composition for a pod cartridge system with effervescent and/or beadlet technology is provided.

According to yet another embodiment, a method for filling a pod with supplements having effervescent and/or beadlet technology is provided.

According to another embodiment, a method for administering supplements to a user, with the supplements containing effervescent and/or beadlet technology is provided.

### BRIEF DESCRIPTION OF THE DRAWINGS

FIGS. 1A and 1B illustrate perspective views of a supplement dispensing machine and a portable dispensing lid, respectively, according to examples and configured for use with the present disclosure;

FIG. 2 is a side sectional view of a pod according to an example and for use with the present disclosure;

FIG. 3 is a schematic illustrating a constituent make-up of a nutritional supplement according to an embodiment;

FIGS. 4A-B illustrate a liquid such as a beverage, prior to and after a supplement is added thereto according to an embodiment;

FIG. 5 is a schematic illustrating a constituent parts of an active ingredient in the form of beadlets according to an embodiment;

FIG. 6 is a schematic illustrating a constituent parts of active ingredient(s), flavoring(s) and/or effervescent agents(s) in the form of beadlets;

FIG. 7 is a schematic, sectional, perspective view of a beadlet according to an embodiment;

FIG. 8 is a perspective view of a pod partially filled with beadlets according to an embodiment;

FIG. 9 is a perspective view of another pod filled with beadlets according to an embodiment;

FIGS. 10A-B illustrate a side sectional view of a pod and a detailed view of the pod according to another embodiment; and

FIGS. 11A-C schematically illustrate a reaction when an effervescent agent is mixed with a liquid according to an embodiment.

### DETAILED DESCRIPTION

As required, detailed embodiments of the present disclosure are provided herein; however, it is to be understood that the disclosed embodiments are merely exemplary and may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present disclosure. Description of constituents in chemical terms refers to the constituents at the time of addition to any combination specified in the description, and does not necessarily preclude chemical interactions among constituents of the mixture once mixed.

As used herein, the terms dietary formula and nutritional supplement, and the abbreviated forms “supplements” and “formulas,” are used more or less interchangeably. Both terms are intended to broadly define any and all types of nutraceuticals, vitamins, minerals, fibers, fatty acids, proteins, amino acids, effervescents, herbal medicines, bodybuilding formulas, pharmaceuticals, therapeutics, medicines, pet products, drugs, treatments and any other like substance that is ingested or non-ingested for useful purposes. Formulas is also intended to broadly include foods for humans, animals and plants.

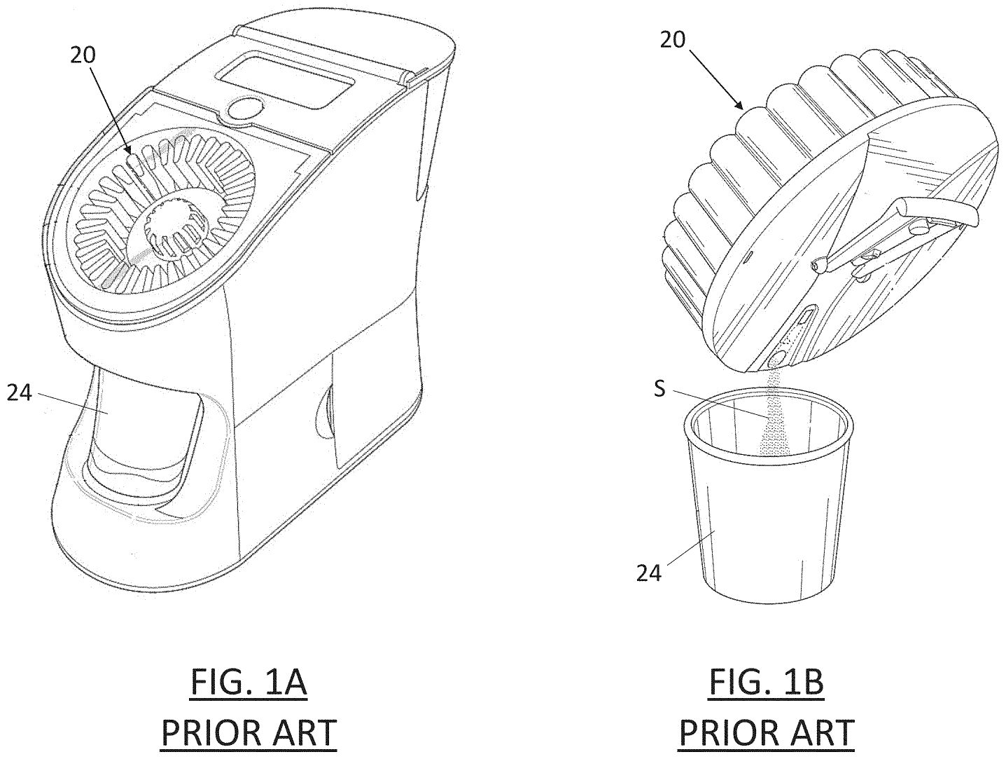

Life Boost, Inc. of Plymouth, Mich., USA, the Applicant of this present invention, has developed a granulated nutritional supplement dispensing machine configured to extract single-serving doses from a multi-serving supplements cartridge for delivery as a drinkable solution, which is marketed under the brand name TESPO. This product is detailed in U.S. Patent Publication No. 2016/0280454, published Sep. 29, 2016, the disclosure of which is hereby incorporated by reference in its entirety herein. An image of the TESPO® machine is shown in FIG. 1A. The TESPO machine utilizes a multi-celled supplements cartridge, or pod, 20 that is automatically indexed so that an unopened serving chamber 22 is presented each time for extracting its supplements. Upon command, a lead serving chamber 22 is pierced so that its powered contents drain into a mixing cup 24 together with a metered quantity of water from an integrated water tank. A vibrator unit assists drainage of the granulated materials from the lead serving chamber. The mixing cup is spun on an inclined turntable to blend the water and supplements into a slurry.

This same company, Life Boost, Inc., has also developed and marketed a light-weight dispensing lid to enable manual extraction of nutritional supplements from the pod 20 for travel applications, which is marketed under the brand name TESPO-GO. This product is detailed in PCT Patent Publication No. WO 2016/126904, published Aug. 11, 2016, and U.S. patent application Ser. No. 16/377,588 filed Apr. 8, 2019, the disclosures of which are hereby incorporated by reference in their entirety herein. An image of the TESPO-GO operatively coupled to a supplements pod 20 is shown in FIG. 1B.

There is a need in the art for an improved formulation for dietary supplements and also improved delivery methods that are amenable to a broadened range of supplement compositions and states (e.g., powders, granules, beadlets/pellets, pills, liquids, etc.). The improved formulation and delivery methods must be conducive to serving-to-serving variability but yet not be susceptible to cross-contamination. The improved formulation and delivery methods should easily accommodate segregation of certain components that are ill-suited for long-term comingled storage but that nevertheless are intended for concurrent consumption in a single dose. Furthermore, the improved formulation and delivery methods must be easy to evacuate with little-to-no external agitation.

Referring to the figures, wherein like numerals indicate like or corresponding parts throughout the several views, a pod is generally shown at 20 throughout the views in various exemplary configurations. The pod 20 has multiple serving chambers 22 arranged in an annular array to accommodate rotary indexing from one serving chamber 22 to the next. In the illustrated examples, all of the serving chambers 22 in the pod 20 are of identical size and shape. Each has a truncated sector-shape radiating from a central drive hub. The drive hub is adapted to interface with a rotary drive shaft like that described, for example, in the aforementioned US 2016/0280454. However, in some contemplated embodiments the serving chambers 22 are of unequal size/shape and in some embodiments the central drive hub is omitted in favor or an external/circumferential drive interface (not shown). The central drive hub may be any type of drive connection or couplings for the transmission of rotational power including, but not limited to, a pass-through hole or stopped hole or coupling, and with or without spline teeth.

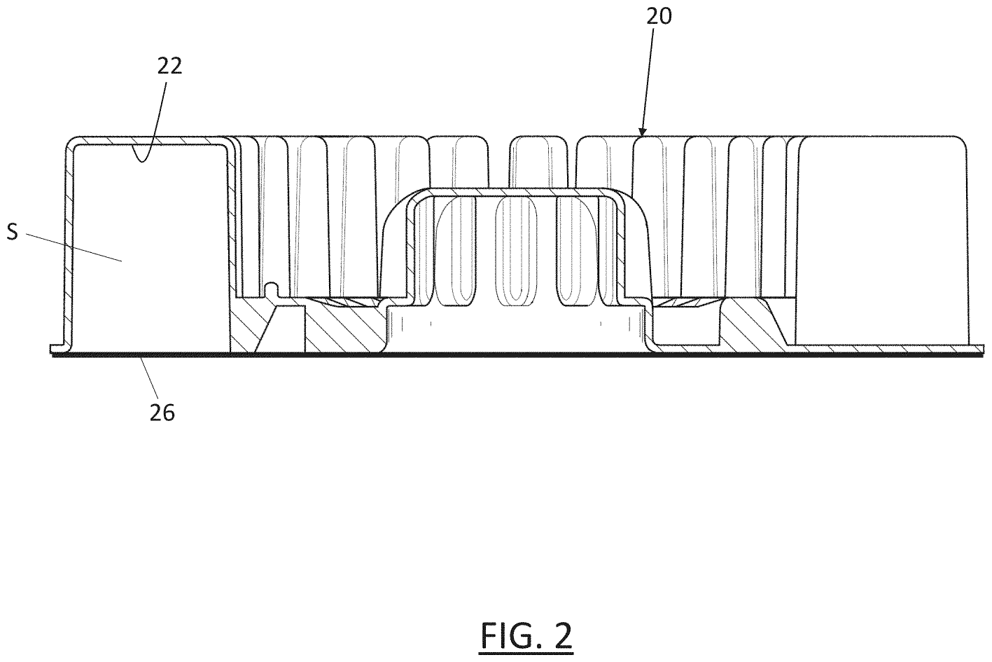

A cross-sectional view of an exemplary pod 20 is shown in FIG. 2. The serving chambers 22 are designed for universal use, in terms of the types and forms of supplements S that can be dispensed. For example, the nutritional supplements S could be of any one or more of the following types: active ingredients, flavoring, special agents, foods (e.g., teas and spices), and so forth. The nutritional supplements S may further include inactive ingredients used as a binding material, and the binding material may further readily dissolve in liquid. In terms of form, the nutritional supplements S capable of use in the pod 20 include, but are not limited to: powders, granules, tablet, beadlets/pellets, and the like. Normally, the supplements S will comprise a formulated blend of active ingredients A and flavoring F. It must be understood that the forms and types and compositions of supplements S mentioned here are for purposes of illustration only, and are not intended to represent exhaustive lists. The top of each serving chamber 22 is closed (or capable of being sealed closed) with a foil membrane 26 or lid or other suitable sealing device.

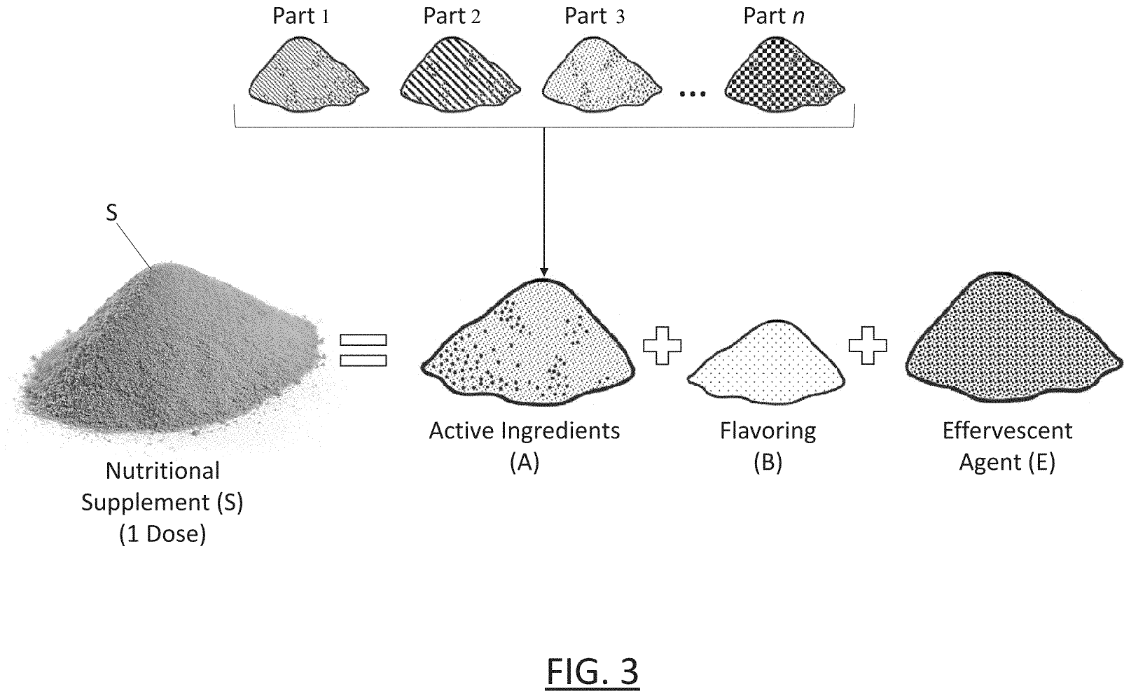

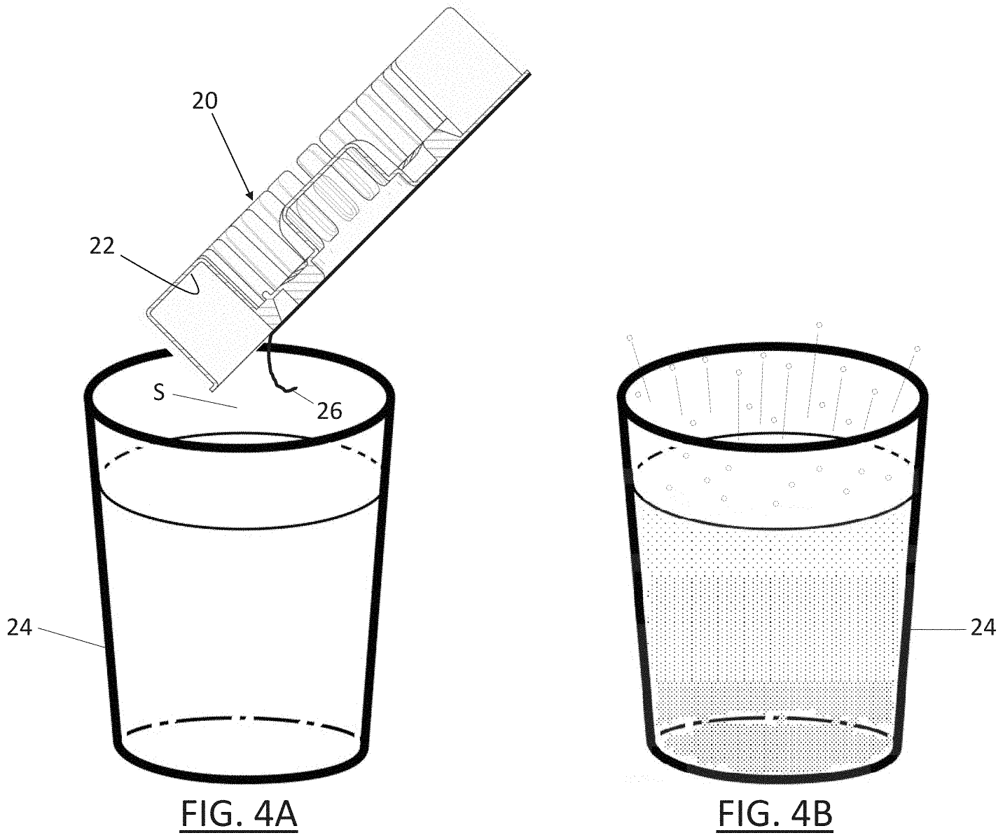

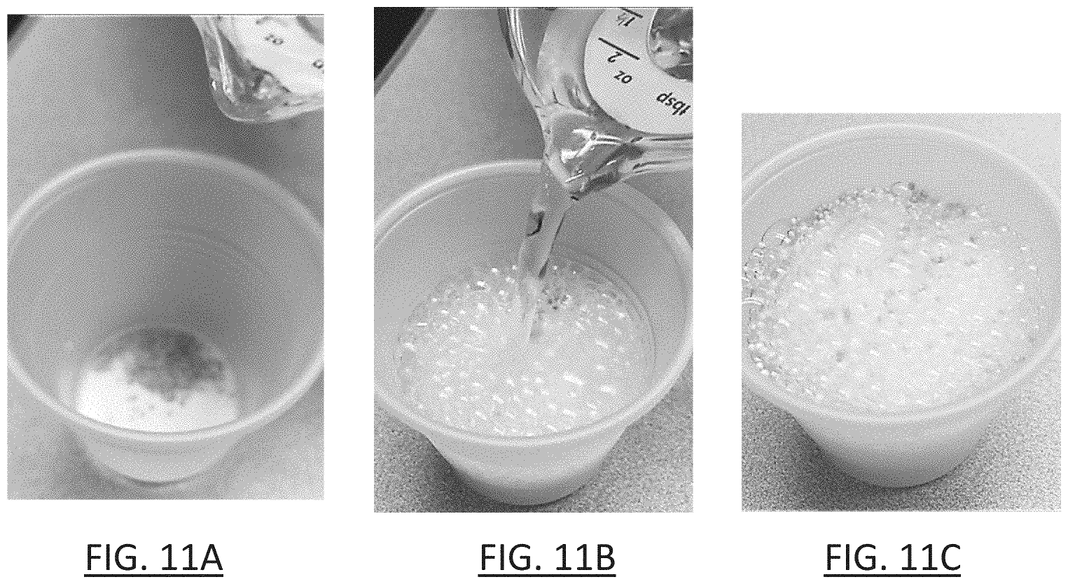

One particularly beneficial attribute of the novel nutritional supplement S composition of this invention is that the supplement composition S includes an effervescent agent E. The constituent make-up of the nutritional supplement S is thus shown illustratively in FIG. 3, comprising one or more active ingredients A, flavoring F and an effervescent agent E. As depicted in FIGS. 4A and 4B, when a supplement S formulated with an effervescent agent E is added to water (or other suitable beverage) prior to consumption, the effervescent agent E causes gas to escape from the aqueous solution with a resulting foaming or fizzing release. FIGS. 11A-11C show a typical reaction when effervescent agent E is exposed to liquid water. According to a non-limiting example, the effervescent agent may be a solid compound that includes an acid and a base. When the effervescent agent is mixed with a liquid, it dissolves and the chemicals are released and can react to form a gas-phase within the liquid. This reaction and resulting gas-phase causes bubbles and effervescence in the liquid. In a further non-limiting example, the effervescent agent may be a solid compound that includes a citric acid and a bicarbonate, and forms carbon dioxide gas when mixed with a liquid containing water.

The supplement S mixed with an effervescent agent E yield numerous benefits and advantages. One subjective but nevertheless genuine benefit is a pleasant bubbly sensation in the user's mouth. For many people, a carbonated or effervescing beverage is simply more enjoyable to drink than a still beverage of the same composition. However, the effervescent agent E also provides several objective benefits. For example, the energetic action induced by the effervescent agent E causes the other active ingredients A and/or flavoring F to remain suspended in the water instead of rapidly sinking to the cup 24 bottom or clinging to the cup 24 sides from surface tension. As a result, less of the active ingredient A and/or flavoring F is likely to remain as residue in the cup 24. Particularly in the case of active ingredients A, a greater percentage will be consumed with each dose due to the incorporation of effervescent agent E by improved dispersion and absorption. (I.e., because of the gas and fizzing effect, the liquid becomes more readily absorbed by the consumer.) Furthermore, the effervescent agent E tends to mask undesirable tastes or flavors from individual or premixed active ingredients A, thus requiring less (or no) use of flavoring F.

Some of the many benefits of an effervescent agent E may include: Multi-sensory consumer experience; Visually interesting dissolution; Fizzing; Color and Color changing; Sound; Excellent aroma dispersion; Disperses functional/active ingredients uniformly in solution; Pre-measured unit doses are easy for consumers to use; Anhydrous product form provides stabilizing environment for sensitive ingredients; and Dense products are economical to transport. Additional details about the benefits of effervescence in “Effervescent Technology for Today's Pharmaceuticals and Dietary Supplements” by Allen Rau, published in the July 2016 edition of Tablets & Capsules.

As suggested in connection with FIG. 3, the active ingredient A can itself be a compound—that is a blend of two or more different elements. These different ingredients are labeled generically as Part 1, Part 2, Part 3 . . . Part n to suggest that the number of separate elements can be any number. In various embodiments, n may be one, or may be greater than one, such as five, ten, twenty, fifty, or more. Each of the n elements may be provided with the same or varying amounts (mass, weight, or volume) within the compound. According to one non-limiting example, n=23 and the active ingredients A for one supplements S dose may be formulated as a twenty-three part or element compound as shown below in the table:

| | Amount per serving | |
|---|---|---|
| Vitamin A (as Vitamin A Palmitate) | 2500 | IU |
| Vitamin C (Ascorbic Acid) | 100 | mg |
| Vitamin D3 (as Cholecalciterol) | 800 | IU |
| Vitamin E (as D-Alpha Tocopheryl Succinate) | 15 | IU |
| Vitamin K1 (as Phytonadione) | 80 | mcg |
| Vitamin B1 (as Thiamin HCl) | 1.5 | mg |
| Vitamin B2 (as Riboflavin-5-Phosphate) | 1.7 | mg |
| Niacin (as Niacinamide) | 10 | mg |
| Vitamin B6 (as Pyridoxal-5-Phosphate) | 2 | mg |
| Folate (as L-Methylfolate) (Metafolin ®) | 400 | mcg |
| Vitamin B12 (as Methylcobalamin) | 100 | mcg |
| Biotin | 300 | mcg |
| Pantothenic Acid (as Calcium Pantothenate) | 10 | mg |
| Iodine (as Potassium Iodide) | 150 | mcg |
| Magnesium (as Magnesium Lactate) | 20 | mg |
| Selenium (as Selenomethionine) | 20 | mcg |
| Manganese (as Manganese Amino Acid Chelate) | 2 | mg |
| Chromium (as Chromium Polynicotinate) | 120 | mcg |
| Molybdenum (as Sodium Molybdate) | 37 | mcg |
| Boron (as Boron Citrate) | 80 | mcg |
| CoQ10 (All-Q ™) | 25 | mg |
| Lutein (FloraGLO ®) | 10 | mg |
| Zeaxanthin (OPTISHARP ®) | 2 | mg |

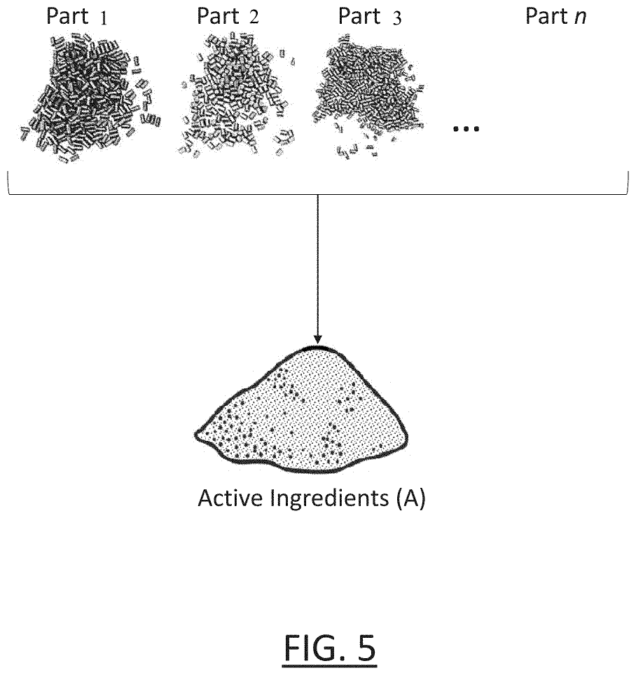

Turning now to FIGS. 5-7, the constituent Parts 1 to n of the active ingredients A are each manufactured in the form of beadlets. For the avoidance of doubt, the term “beadlet” is intended to very broadly include all forms of micro-beads and pellets and extrusions and other manner of intentionally-shaped compositions. Beadlets may range in size from below 0.2 mm to over 5.0 mm in diameter, and within a range between 0.2 to 5.0 mm. Beadlets of the different constituent parts may be uniform or vary in shape, size, weight, color or may vary within the range of about 0.2 mm to about 5.0 mm.

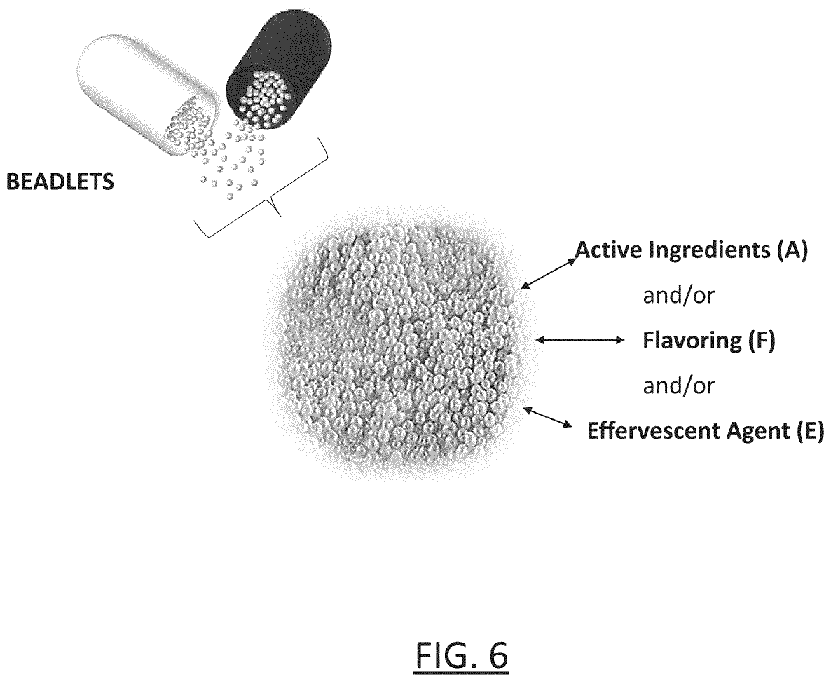

Moreover, the use of beadlets is not limited to the active ingredients A. As shown in FIG. 6, the flavoring F and/or the effervescent agent E can also be fabricated as beadlets in addition to the active ingredients. Thus, any combination of some or all of the supplement S composition can be in the form of beadlets.

Beadlets offer many advantages within the context of dietary supplements and/or pharmaceuticals consumed in measured doses by mixture with water (or other liquid). One such advantage is that because of the reliably-consistent flow properties of the beadlet form, the active ingredients A can be very accurately processed through filling machinery doses on size, weight, volume or individual ingredient.

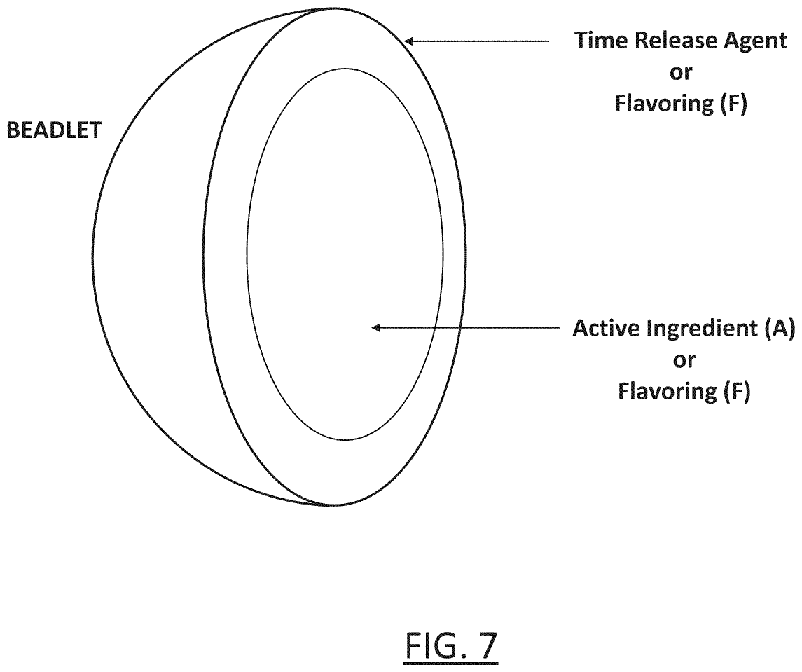

Another advantage of beadlet technology is described in connection with FIG. 7. In particular, current beadlet technology enables the core active ingredient A or flavoring F to be coated with a shell of non-water or water-soluble coating (fat or LMP, lipid multi-particulates). Commonly, the shell comprises a time-release agent, however the shell could instead be the flavoring F (covering an active ingredient A core).

In the example of a time-release coating, the same beadlets within a single supplements S dose could be entirely uncoated, some could be coated with a thin layer of time-release agent, and still other beadlets coated with a thick layer of time-release agent. In this manner, a controlled time release can be achieved over a long period of time after consumption. Time release in the beadlet form the active ingredient A benefits by being released in different time intervals in the user's digestion track to release different Active ingredients (A) at different times of the day or for adsorption over an extended time period. In another example associated with FIG. 7, some or all of the beadlets within a single supplements S dose could be coated with a flavoring F to help mask otherwise undesirable tastes or flavors from the core active ingredients A.

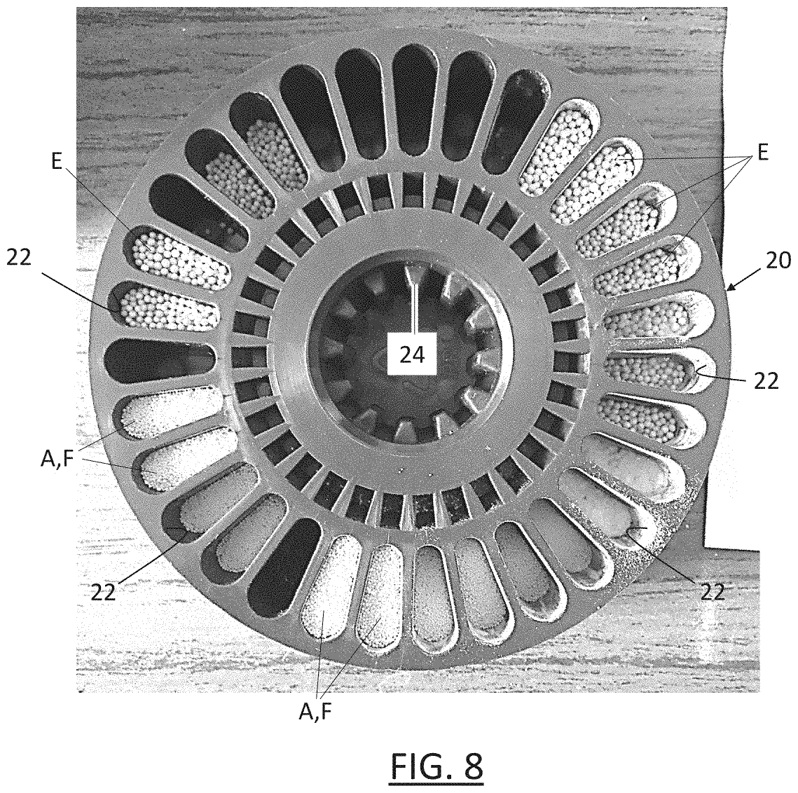

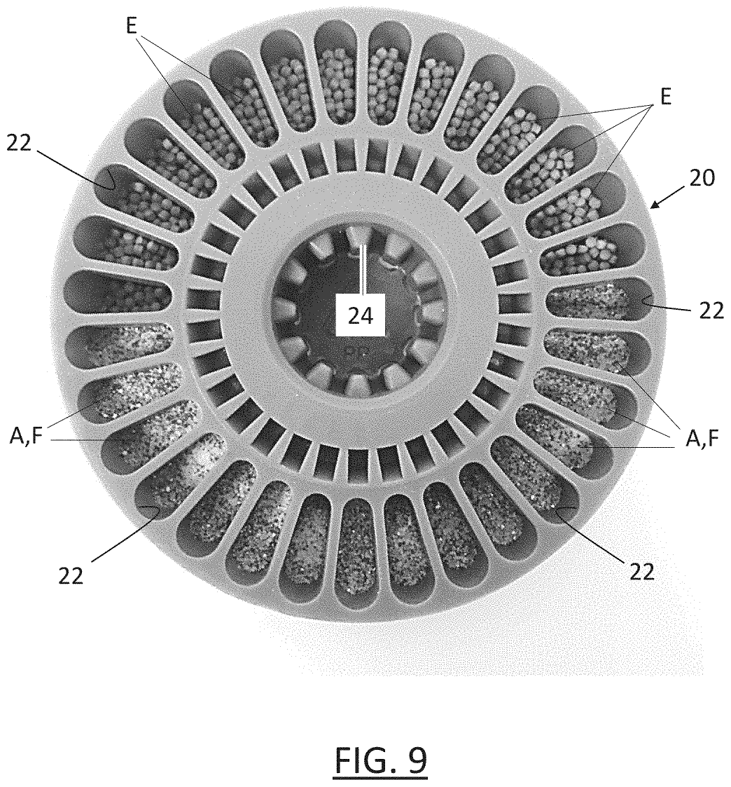

Turning now to FIGS. 8 and 9, views of a filled (or partially-filled) pod 20 are shown without a foil membrane seal 26. In some situations, it may be desirable to segregate certain components within the compounded supplements S that may be ill-suited for long-term comingled storage but that nevertheless are intended for concurrent consumption in a single dose. For one example, if a dose is composed of Part A ingredients and Part B ingredients, however Part B ingredients will cause clumping in Part A when stored together or otherwise provoke some other adverse interaction, then Part A ingredients can be contained in separate serving chambers 22 from the Part B ingredients. FIGS. 8 and 9 illustrate this principle by identifying the effervescent agent E as the ingredient that must be segregated from the active ingredient/flavoring A/F prior to use. At the time of dispensing, a serving chamber 22 containing a Part A/F ingredient will be first evacuated into a mixing cup, container, bottle (not shown), and immediately thereafter the Part E ingredient is evacuated into the same mixing cup so that all three Parts A/F and E are consumed in the same dose. Of course, the order could be reversed with the effervescent Part E first evacuated. The order of adding water or another liquid could be reversed as well: e.g., water added first then powder, or powder first then water.

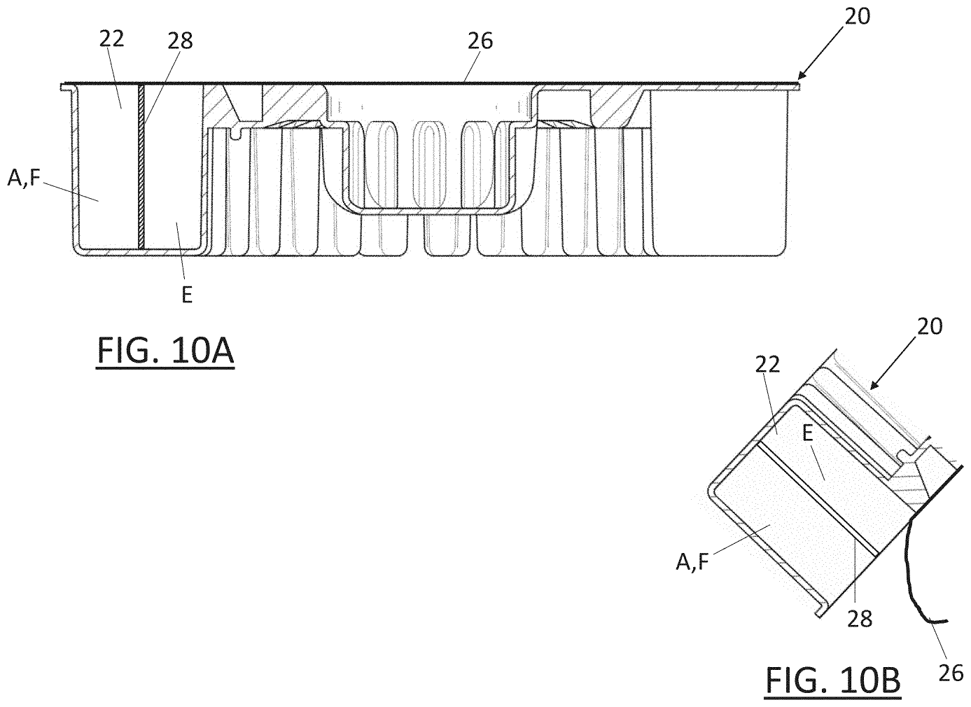

Alternatively, the serving chamber 22 can itself be segregated with a partition 28, as shown in FIGS. 10A and 10B. The partition 28 can be used to maintain physical separation between the Part A/F and Part E ingredients (continuing the previous example). As illustrated in these highly simplified figures, Parts A/F and E are maintained separate until the foil membrane 26 (or lid, etc.) is opened, at which time Parts A/F and E are simultaneously evacuated.

In another contemplated embodiment, the effervescent agent E, active ingredients A and flavoring F could be consolidated into a monolithic tablet contained in the serving chamber 22. This “all in one tablet” option may be loosely compared to certain effervescent antacid and pain reliever products such as ALKA-SELTZER.

In summary, the supplement S may be composed of the following combinations of flavoring F, active ingredients A and effervescent agent E. Although preferably these elements are in beadlet form, the principles of this invention will apply also to various granulated size ingredients, powders, gel tablets or capsules and even pills. The supplements S formula is held inside individual serving cells 22 in a multi-cell container or pod 20. Each pod 20 has individual serving cells 22 that can be filled with a combination of these ingredients A, F and E to create individual supplement formulas S in individual cells 22 in individual pod 20. These combinations of ingredients can be ingested in many ways, they can be added to various liquids and smoothies, consumed stand alone or on top of other foods. When added to a liquid or liquid added to formula S the combination of ingredients of said formula S will react and release air bubbles that mix the formula, suspend the formula grains, beadlets, tablet off the cup bottom and provide a mouthfeel for a consumer drinking the liquid and formula combined.

In another contemplated variation, the effervescent could be provided in the form of a tablet. Filling of effervescent could be confined to separate air tight hoppers, where 1-part effervescent is added to each serving chamber 22 followed by a second hopper air tight filled with the 2nd part effervescent added to the same pod cells in the same dosed amount. All cells may be filled with exactly the same volumes of ingredients; however, this is not limited to filling each cell with completely different volumes of ingredients and thus different volumes of 1 part effervescent and 2nd part effervescent.

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure.

What is claimed is:

1. A pod for dispensing a nutritional supplement, the pod comprising:
a housing defining a plurality of serving chambers arranged in an annular array and surrounding a drive hub;
a nutritional supplement provided within at least one of the plurality of serving chambers, the nutritional supplement comprising at least one active ingredient and an effervescent agent to form a gas when the nutritional supplement is dispensed from the pod and dissolved in a liquid; and
at least one sealing member connected to the housing and extending over the plurality of serving chambers, the at least one sealing member being removable to open a selected one of the plurality of serving chambers to dispense supplement therein.

2. The pod of claim 1 further comprising a partition positioned within one of the plurality of serving chambers, the partition providing physical separation between the active ingredient and the effervescent agent within the serving chamber until the serving chamber is opened and dispensed.

3. The pod of claim 1 wherein the at least one active ingredient of the nutritional supplement is provided within one of the plurality of serving chambers, and the effervescent agent of the nutritional supplement is provided in another of the plurality of serving chambers such that the effervescent agent is segregated from the at least one active ingredient within the pod.

4. The pod of claim 1 wherein the at least one active ingredient comprises a Part A active ingredient and a Part B active ingredient; and
wherein the Part A active ingredient is provided within one of the plurality of serving chambers, and the Part B active ingredient is provided in another of the plurality of serving chambers such that the Part B active ingredient is segregated from the Part A active ingredient within the pod.

5. The pod of claim 1 wherein the nutritional supplement is formed as a plurality of beadlets.

6. The pod of claim 5 wherein each beadlet of the plurality of beadlets is formed with a shell encapsulating a core, the core being provided by the at least one active ingredient, and the shell being provided by a flavoring.

7. The pod of claim 5 wherein a first portion of the plurality of beadlets is formed as an uncoated core, and a second portion of the plurality of beadlets is formed with a shell encapsulating a core; and
wherein the cores of each of the first and second portions contains the at least one active ingredient.

8. The pod of claim 7 wherein the shell of each beadlet of the second portion of beadlets has a first thickness;
wherein a third portion of the plurality of beadlets is formed with another shell encapsulating a core, the another shell having a second thickness greater than the first thickness; and
wherein the cores of each of the first, second, and third portions contains the at least one active ingredient.

9. The pod of claim 1 wherein the effervescent agent further comprises a solid compound that includes an acid and a base.

10. The pod of claim 1 wherein the effervescent agent further comprises a citric acid and a bicarbonate, and forms carbon dioxide gas when mixed with a liquid containing water.

11. The pod of claim 1 wherein the at least one active ingredient is formed as a plurality of beadlets.

12. The pod of claim 1 wherein the nutritional supplement further comprises a flavoring.

13. The pod of claim 12 wherein the nutritional supplement is formed as a monolithic tablet containing the effervescent agent, the active ingredient, and the flavoring.

14. The pod of claim 1 wherein the active ingredient of the nutritional supplement comprises at least one of a vitamin, a mineral, a fiber, a fatty acid, a protein, an amino acid, an herbal medicine, a bodybuilding supplement, a pharmaceutical, a therapeutic, a medicine, a drug, and a treatment.

15. A method of providing a nutritional supplement comprising:
providing a housing defining a plurality of serving chambers arranged in an annular array and surrounding a drive hub;
providing a nutritional supplement into at least one of the plurality of serving chambers of the pod, the nutritional supplement comprising at least one active ingredient and an effervescent agent; and
sealing the nutritional supplement within the at least one of the plurality of serving chambers of the housing by connecting at least one sealing member to the housing to form a pod, the at least one sealing member extending over the plurality of serving chambers and being removable to open a selected one of the plurality of serving chambers to dispense supplement therein.

16. The method of claim 15 further comprising dispensing the nutritional supplement into a liquid by opening the at least one sealing member such that the nutritional supplement from the selected one of the plurality of serving chambers is dispensed from the pod and into the liquid, wherein the effervescent agent forms a gas when the nutritional supplement is dispensed from the pod and dissolved in the liquid.

17. The method of claim 15 wherein the nutritional supplement is provided as a plurality of beadlets, with a first portion of the plurality of beadlets is formed as an uncoated core, and a second portion of the plurality of beadlets is formed with a shell encapsulating a core, wherein the cores of each of the first and second portions contains the at least one active ingredient.

18. The method of claim 15 further comprising providing a partition in the housing, the partition positioned within and dividing one of the plurality of serving chambers, the partition providing physical separation between the active ingredient and the effervescent agent within the serving chamber until the serving chamber is opened and dispensed.

19. The method of claim 15 wherein the nutritional supplement is provided with the at least one active ingredient into one of the plurality of serving chambers, and the effervescent agent of the nutritional supplement into another of the plurality of serving chambers such that the effervescent agent is segregated from the at least one active ingredient within the pod; and
wherein the method further comprises opening both the one of the plurality of serving chambers and the another of the plurality of serving chambers when dispensing the nutritional supplement.

20. The method of claim 15 wherein the at least one active ingredient comprises a Part A active ingredient and a Part B active ingredient;
wherein nutritional supplement is provided with the Part A active ingredient into one of the plurality of serving chambers, and the Part B active ingredient into another of the plurality of serving chambers such that the Part B active ingredient is segregated from the Part A active ingredient within the pod; and
wherein the method further comprises opening both the one of the plurality of serving chambers and the another of the plurality of serving chambers when dispensing the nutritional supplement.
